(19) United States
(12) Patent Application Publication
(10) Pub. No.: US 2013/0131390 A1
(43) Pub. Date: May 23, 2013

(54) PROCESS FOR THE PRODUCTION OF SUBSTITUTED ELECTRON RICH DIPHENYLACETYLENES

(71) Applicants: Ulla Letinois, Saint-Louis (FR); Werner Bonrath, Freiburg (DE)

(72) Inventors: Ulla Letinois, Saint-Louis (FR); Werner Bonrath, Freiburg (DE)

(21) Appl. No.: 13/386,385

(22) PCT Filed: Jul. 21, 2010
(86) PCT No.: PCT/EP10/60569

§ 371 Date: Sep. 14, 2012

(30) Foreign Application Priority Data
Jul. 22, 2009 (EP) 09166121.5

(51) Int. Cl. C07C 41/30; C07C 41/20

(52) U.S. Cl. CPC C07C 41/30; C07C 41/20
USPC 568/646

(57) ABSTRACT

The present invention relates to an improved process of production of substituted diphenylacetylenes (tolanes) of formula (I) which are starting materials for production of stilbenes products.

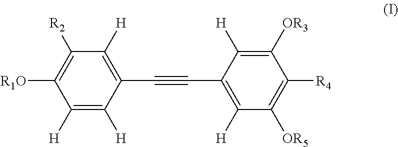

The present invention relates to an improved process for the production of substituted electron rich diphenylacetylenes (tolanes), which are starting materials for the production of stilbenes.

The present invention relates to the process for production of a compound of formula (I)

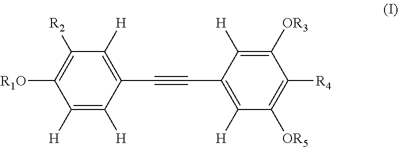

These tolanes are characterized in that at least one of the phenyl rings is substituted by at least two substituents. The compounds according to formula (I) can be used as starting material for the production of the corresponding stilbenes. Some of the stilbenes are compounds with interesting pharmacological properties.

Among these pharmacological products are for example combretastatin A-4 (compound of formula (1)) and resveratrol (compound of formula (2)).

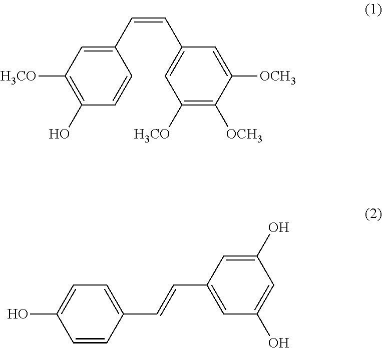 [SMILES: COc1cc(/C=C\c2cc(OC)c(OC)c(OC)c2)ccc1O ; Oc1ccc(/C=C/c2cc(O)cc(O)c2)cc1]

Combretastatin A-4 is potent in regard to tubulin binding ability and it is also cytotoxic.

Resveratrol is a well known nutritional supplement with healthy properties.

Both compounds can be extracted from natural sources. For an industrial product extraction from natural sources is not suitable at all. Therefore these products are usually produced synthetically. Therefore there is always a need to simplify and optimize such processes of production or to provide new syntheses for the production.

A few syntheses are known from prior art from which tolanes are obtainable. Mostly all of these syntheses comprise some kind of catalytic methods. In principle two kinds of catalyst are used:
(i) homogeneous catalysts (these act in the same phase than the reactants)
(ii) heterogeneous catalysts (these act in a different phase than the reactants)

For the production of tolanes according to formula (I) only synthesis using homogeneous catalytic systems are described. One of the most prominent ones is the Sonogashira coupling in which usually palladium catalysts under homogeneous conditions are used. Such a catalyst system is usually used in combination with a base and a halide salt of copper(I).

The Sonogashira coupling reaction, wherein a homogeneous catalyst is used has some disadvantages. For example:
the catalytic system has to be separated from the reaction products
it is hardly possible to remove all the catalyst
the reaction is carried out under an inert gas atmosphere
the reusability of the catalyst is not very good
when recycling is carried out, decreased yields are often obtained
the product is often palladium and copper contaminated

The goal of the present invention was to find a process for the production of compounds of electron rich tolanes of formula (I), which does not have the disadvantages as mentioned above. Surprisingly it was found that when a heterogeneous catalytic system is used, the above mentioned disadvantages are overcome.

Therefore the present invention relates to a process for the production of compounds of formula (I)

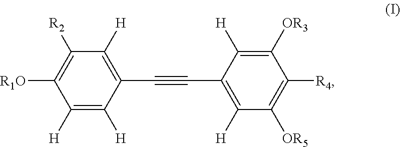

in which
R₁ is H; linear, branched or cyclic C₁-C₆ alkyl; tetrahydropyryl or —CH₂-phenyl; preferably H; —CH₃; —CH₂CH₃, or —CH₂-phenyl; more preferably H; —CH₃; or —CH₂CH₃;
R₂ is H or OR′₂, wherein R′₂ is H; linear, branched or cyclic C₁-C₆-alkyl or —CH₂-phenyl; preferably R₂ is H or OR′₂, wherein R′₂ is —CH₃ or —CH₂CH₃;
R₃ is H; linear, branched or cyclic C₁-C₆ alkyl; tetrahydropyryl or —CH₂-phenyl; preferably H; —CH₃; —CH₂CH₃, or —CH₂-phenyl; more preferably H; —CH₃, or —CH₂CH₃;
R₄ is H or OR′₄, wherein R′₄ is H; linear, branched or cyclic C₁-C₆-alkyl or —CH₂-phenyl; preferably R₄ is H or OR′₄, wherein R′₄ is —CH₃ or —CH₂CH₃; and
R₅ is H; linear, branched or cyclic C₁-C₆ alkyl; tetrahydropyryl or —CH₂-phenyl; preferably H; —CH₃; —CH₂CH₃, or —CH₂-phenyl; more preferably H; —CH₃, or —CH₂CH₃;
   wherein a compound of formula (IIa) or (IIb)

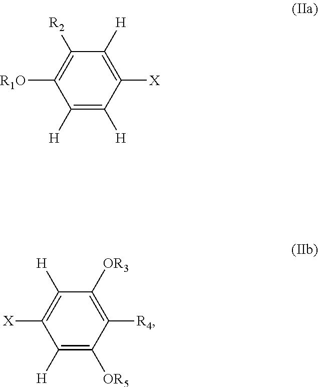

in which
the substituents R₁, R₂, R₃, R₄ and R₅ have the same meanings as defined for formula (I) and
X is —I; —Br; —Cl; or —N₂,
   is reacted with a compound of formula (IIIa) or (IIIb)

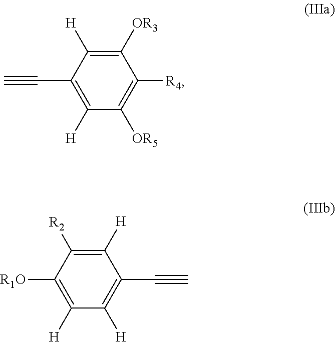

in which
the substituents R₁, R₂, R₃, R₄ and R₅ have the same meanings as defined for formula (I),
   characterized in that
   a heterogeneous catalytic system is used.

The linear, branched and cyclic C₁-C₆-alkyl groups (in the definition of R₁, R₂, R′₂, R₃, R₄, R′₄ and R₅) can also be substituted. Suitable substituents are C₁-C₄alkoxy (preferably —OCH₃ and —OCH₂CH₃) and aryl. In case one or more linear, branched and cyclic C₁-C₆-alkyl groups are substituted by at least one substituent, then the substituent is chosen from the group consisting of C₁-C₄alkoxy (preferably —OCH₃ and —OCH₂CH₃) and aryl.

The —CH₂-phenyl groups (in the definition of R₁, R₂, R′₂, R₃, R₄, R′₄ and R₅) can also be substituted. Suitable substituents are C₁-C₄ alkyl (preferably —CH₃ and —CH₂CH₃) and aryl. In case one or more —CH₂-phenyl groups are substituted by at least one substituent, then the substituent is chosen from the group consisting of C₁-C₄ alkyl (preferably —CH₃ and —CH₂CH₃); C₁-C₄alkoxy (preferably —OCH₃ and —OCH₂CH₃) and aryl.

A preferred embodiment of the present invention is a process for the production of a compound of formula (I) as described above, wherein a compound of formula (IIa) is reacted with a compound of formula (IIIa).

Another preferred embodiment of the present invention is a process for the production of a compound of formula (I) as described above, wherein a compound of formula (IIb) is reacted with a compound of formula (IIIb).

Preferred compounds, which are produced according to the process of the present invention, are compounds of formula (Ia)

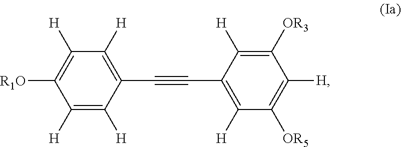

wherein
R₁, R₃ and R₅ are independently from each other H; linear, branched or cyclic C₁-C₆-alkyl; tetrahydropyryl or —CH₂-phenyl.

Preferably R₁, R₃ and R₅ are independently from each other H; —CH₃ or —CH₂CH₃. More preferably R₁, R₃ and R₅ are H. Further more preferably R₁, R₃ and R₅ are CH₃.

The linear, branched and cyclic C₁-C₆-alkyl groups (in the definition of R₁, R₃ and R₅) can also be substituted. Suitable substituents are C₁-C₄alkoxy (preferably —OCH₃ and —OCH₂CH₃) and aryl. In case one or more linear, branched and cyclic C₁-C₆-alkyl groups are substituted by at least one substituent, then the substituent is chosen from the group consisting of C₁-C₄alkoxy (preferably —OCH₃ and —OCH₂CH₃) and aryl.

The —CH₂-phenyl groups (in the definition of R₁, R₃ and R₅) can also be substituted. Suitable substituents are C₁-C₄ alkyl (preferably —CH₃ and —CH₂CH₃) and aryl. In case one or more —CH₂-phenyl groups are substituted by at least one substituent, then the substituent is chosen from the group consisting of C₁-C₄ alkyl (preferably —CH₃ and —CH₂CH₃); C₁-C₄alkoxy (preferably —OCH₃ and —OCH₂CH₃) and aryl.

It is obvious that the substituents in the formula (II) and (III) are defined in analogy to the ones in the compound of formula (Ia).

Further preferred compounds, which are produced according to the process of the present invention are compounds of formula (Ib)

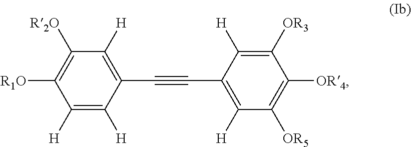

wherein
R₁, R′₂, R₃, R′₄ and R₅ are independently from each other H; linear, branched or C₁-C₆-alkyl; tetrahydropyryl or —CH₂-phenyl.

Preferably R′₂, R₃, R₄′, and R₅ are independently from each other —CH₃ or —CH₂CH₃, and R₁ is H. More preferably R′₂, R₃, R′₄ and R₅ are independently from each other —CH₃ or —CH₂CH₃, and R₁ is H. Most preferably R₁ is H and R′₂, R₃, R′₄ and R₅ are —CH₃.

The linear, branched and cyclic C₁-C₆-alkyl groups (in the definition of R₁, R′₂, R₃, R′₄ and R₅) can also be substituted. Suitable substituents are C₁-C₄alkoxy (preferably —OCH₃ and —OCH₂CH₃) and aryl. In case one or more linear, branched and cyclic C₁-C₆-alkyl groups are substituted by at least one substituent, then the substituent is chosen from the group consisting of C₁-C₄alkoxy (preferably —OCH₃ and —OCH₂CH₃) and aryl.

The —CH₂-phenyl groups (in the definition of R₁, R₂, R′₂, R₃, R₄, R′₄ and R₅) can also be substituted. Suitable substituents are C₁-C₄ alkyl (preferably —CH₃ and —CH₂CH₃) and aryl. In case one or more —CH₂-phenyl groups are substituted by at least one substituent, then the substituent is chosen from the group consisting of C₁-C₄ alkyl (preferably —CH₃ and —CH₂CH₃); C₁-C₄alkoxy (preferably —OCH₃ and —OCH₂CH₃) and aryl.

It is obvious that the substituents in the starting products (II) and (III) are defined in analogy to the ones in the compound of formula (Ib).

The process for the production of the present invention is catalyzed by a heterogeneous catalytic system. The catalytic system is a heterogeneous system with catalysts on a carrier for example, Pd/BaSO₄, Pd/CaCO₃, Pd/Al₂O₃, Pd/TiO₂, Pd/SiO₂, Pd/ZnO, Pd/C with palladium loadings of 1-12 weight-% (wt-%), preferred 3-10 wt-%, based on the total weight of the catalytic system. The catalyst has a surface area (BET) of 5-400 m²/g, preferably 10-250 m²/g. These catalysts are known from the prior art and can therefore be prepared accordingly. Usually such catalytic systems are commercially available. In the process according to the present invention palladium on charcoal (Pd/C) is a preferred heterogeneous catalytic system.

The reaction according to the present invention is carried out in polar organic solvents, preferred are non-protic solvents, such as DMF, NMP, triethylamine and pyrrolidine. Optionally a base can be added to the solvent as well as ligands like triarylphosphines, trialkylphoshines or aminoethanol. It is obvious that also solvent mixtures can be used.

A suitable reaction temperature for the process of production of compounds of formula (I) is from 25° C.-150° C., preferred 50° C.-120° C.

The compounds of formula (I) as described and defined above are used for the manufacture of the corresponding stilbenes (formula (IV)). Such a transformation can be done according to reduction processes known from the prior art.

But surprisingly a new and improved way for the synthesis of electron rich stilbenes from the corresponding tolanes using heterogeneous hydrogenation catalysts has been found. By the term “corresponding”, it is meant that all the substituents in formula (I) and formula (IV) are identical. It is only the triple bond which is transformed into a double bond.

Therefore a further embodiment of the present invention is an inventive hydrogenation of compounds according to formula (I), for the manufacture of compounds of formula (IV)

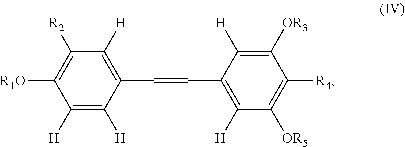

wherein the substituents, R₁, R₂, R₃, R₄ and R₅ have the same meanings as well as the same preferences as defined above.

The compounds of formula (I) can be transformed into the corresponding stilbenes of formula (IV) using reduction processes described in prior art. Such reduction processes are usually using stoichiometric amounts of complex hydrides like NaBH₄ and LiAlH₄. These well known processes have some major drawbacks, for example, the use of complex hydrides causes the formation of stoichiometric amounts of waste.

Surprisingly we found that the compounds of formula (I) can be reduced to the corresponding stilbenes in presence of hydrogen and a heterogeneous catalytic system comprising palladium and lead (Pb) on calcium carbonate. The Pd/Pb content on CaCO₃ varies from 1 to 10 wt-%, based on the total weight of the catalytic system and the Pd/Pb ratio varies from 1:1 to 0.5 to 5.

Very preferred is the hydrogenation using a heterogeneous catalytic system, which leads to the stilbenes of formula (1) and (2).

The H₂ pressure in the hydrogenation process can be from 1.1 bar-10 bar, preferably 1.1 bar-6 bar. The reaction temperature in the hydrogenation process goes from 25° C. to 80° C., preferred is 30-60° C.

The hydrogenation process can be carried out in organic solvents, preferred are polar organic solvents, especially preferred are alcohols from C₂-C₆. It is obvious that also solvent mixtures can be used. But it is also possible to carry out the hydrogenation without any solvents. Such hydrogenations are more preferred than ones using a solvent.

The following examples serve to illustrate the invention. The percentages are expressed in weight percentages and the temperatures are degrees Celsius, if not otherwise defined.

### EXAMPLES

### Example 1

### Synthesis of 4′-Hydroxy-3,5-dimethoxydiphenylacetylene

3 ml of pyrrolidine (99%) were placed in a 10 ml glass tube fitted with a septum, a magnetic stirrer and an argon supply. The solution was degassed with argon for 30 minutes at room temperature. Then (the amount shown in table 1) of 4-halophenole (99%), 10.8 mg (0.04 mmol) triphenylphosphine (97%) and 248.2 mg (1.5 mmol) 1-ethynyl-3,5-dimethoxybenzene were added. Afterwards 35 mg of dried palladium on charcoal (10%) were added.

The mixture was stirred under argon at 85° C. (aluminum block temperature) for 17 hours. The reaction solution was cooled down to room temperature and then 10 ml of ethyl acetate were added. Afterwards the suspension was filtrated with a membrane filter (0.45 μm).

The solution was treated 12 ml of hydrochloric acid solution (10%, 34.3 mmol). Then an extraction was performed by extracting twice with 10 ml of ethyl acetate. The organic solutions were dried with sodium sulfate and afterwards concentrated at 40° C. at 180 mbar. The dark yellow crude material was purified by chromatography with ethyl acetate n-heptane in a ratio of 5:95. The fractions were collected and concentrated at 40° C. and 90 mbar. The isolated fractions were analysed by GC-MS and NMR.

¹H-NMR 300 MHz, d-Chloroform) δ 3.75-3-85 (m, 6H, OCH₃), 4.97 (s, H, OH), 6.35-6.44 (m, 2H, ArH), 6.60 (dd, J=2.28 15.80 Hz, 1H, ArH), 6.74-6.77 (m, 2H, ArH), 7.17-7.20 (m, 2H, ArH). ¹³C-NMR 75 MHz, d-Chloroform) δ 55.40 (OCH₃), 88.32 (C), 92.02 (C), 104.45 (C), 109.22 (CH), 116.65 (2×CH), 115 (C), 124.9 (CH), 130.52 (CH), 160.52 (C), 160.96 (2×C).

TABLE 1
type and concentration of halophenols; yield of 4′-hydroxy-3,5-
dimethoxydiphenylacetylene

| | | 4-Halophenole | |
|---|---|---|---|
| Example | 4-Halophenole | [mg, mmol] | Yield [%] |
| 1a | 4-Chlorophenole | 130.9, 1.02 | 35 |
| 1b | 4-Bromophenole | 174.9, 1.01 | 28 |

### Example 2

### Synthesis of 3,4′,5-trimethoxy-Z-stilbene

0.25 g of 3,4′,5-trimethoxydiphenylacetylene (99%), 25 mg of palladium on calcium carbonate (5% Pd with 3.5% Pb) were placed with 20 g of ethanol (99%) in a 37 ml glass flask. The glass-liner was closed and stirring was started with 500 rpm. The autoclave was flushed three times with 5 bara N₂. The stirrer was turned off. The autoclave was pressurized with 5 bara H₂ for 10 minutes for pressure check. The pressure was released. The stirrer was turned on to 1000 rpm and the autoclave was heated to 60° C. internal temperature. The autoclave was pressurized with 2 bara H₂ and the stirrer was set to 1000 rpm. The reaction mixture was stirred under 2 bara H₂ at 60° C. for 3.3 minutes. Then the autoclave was opened. The content was sucked, filtrated over a 0.45 μm filter and washed with 4 ml of ethanol.

The mixture was concentrated at 40° C. and 120 mbar. The isolated crude product was analyzed by GC/MS and NMR. The total yield calculated with GC/MS was 45%.

GC/MS: Retention time: 19.46 min, Area %: 56.0%; M: M+ 270, 239, 255.

¹H-NMR 300 MHz, d-Chloroform) δ 3.66 ppm (s, 6H, OCH₃), 3.77 ppm (s, 3H, OCH₃), 6.31 ppm (t, J=2.2 Hz, 1H, CH), 6.43 ppm (d, J=2.2 Hz, 3H, ArH), 6.48 (dd, J=14.5, J=12 Hz 1H, H), 6.75-6.77 ppm (m, 2H, ArH), 7.20-7.22 ppm (m, 2H, ArH). ¹³C-NMR 75 MHz, d-Chloroform) δ 55.22 (3×OCH₃), 99.69 (CH), 106.64 (2×CH), 113.55 (2×CH), 128.70 (CH), 129.58 (C), 130.17 (CH), 130.29 (2×CH), 139.50 (C), 158.77 (C), 160.59 (2×C).

### Example 3

### Synthesis of 3,4′,5-trimethoxydiphenylacetylene

5 ml of pyrrolidine (99%) were placed in a 10 ml glass tube fitted with a septum, a magnetic stirring bar and an argon supply. The pyrrolidine was degassed with argon for 30 minutes at room temperature. Afterwards 240 mg of 4-iodoanisol (98%, 1.01 mmol, 1.00 eq.), 12 mg of triphenylphosphine (97%, 0.044 mmol, 0.044 eq.), 40 mg of palladium on charcoal (10%, 0.038 mmol, 0.037 eq.) and finally 249.3 mg of 1-ethynyl-3,5-dimethoxybenzene (98%, 1.51 mmol, 1.50 eq.) were added to the presented pyrrolidine.

The mixture was stirred under argon at 85° C. (aluminum block temperature) for 17 hours. The reaction solution was cooled to room temperature and then 10 ml of ethyl acetate were added. Afterwards the suspension was filtrated with a membrane filter (0.45 μm).

The solution was treated with 20 ml of a saturated ammonium chloride solution. Then an extraction was performed by extracting twice with 20 ml of ethyl acetate. The organic solutions were dried with sodium sulfate and afterwards concentrated at 40° C. and 180 mbar. The dark yellow crude material was purified by chromatography with ethyl acetate and n-heptane in a ratio of 5:95. The product containing fractions were collected and concentrated at 40° C. and 90 mbar. The purified product was analyzed by GC-MS and NMR. The yield was 75% based on the 4-iodoanisole.

GC/MS: Retention time: 21.61 min, Area %: 99.10%; M: M+268, 253, 225, 210, 195, 182, 167, 152, 139.

¹H-NMR 300 MHz, d-Chloroform) δ 3.82 ppm (s, 6H, OCH₃), 3.85 ppm (s, 3H, OCH₃), 6.47 (t, J=2.31 Hz, 1H, ArH), 6.70 ppm (d, J=2.31 Hz, 2H, ArH), 6.88-6.89 ppm (m, 2H, ArH), 7.48-7.52 ppm (m, 2H, ArH). ¹³C-NMR 75 MHz, d-Chloroform) δ 55.30 (OCH₃), 56.42 (2×OCH₃), 88.10 (C), 89.00 (C), 101.57 (C), 109.22 (2×CH₃), 114.02 (2×CH₃), 115.20 (C), 124.91 (CH), 133.12 (2×CH), 159.71 (C), 160.55 (2×C).

1. A process for the production of compounds of formula (I) 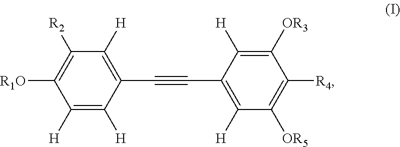
in which
R₁ is H; linear, branched or cyclic C₁-C₆ alkyl; tetrahydropyryl or —CH₂-phenyl;
R₂ is H or OR′₂, wherein R′₂ is H; linear, branched or cyclic C₁-C₆-alkyl or —CH₂-phenyl;
R₃ is H; linear, branched or cyclic C₁-C₆ alkyl; tetrahydropyryl or —CH₂-phenyl;
R₄ is H or OR′₄, wherein R′₄ is H; linear, branched or cyclic C₁-C₆-alkyl or —CH₂-phenyl; and
R₅ is H; linear, branched or cyclic C₁-C₆ alkyl; tetrahydropyryl or —CH₂-phenyl;
wherein a compound of formula (IIa) or (IIb) 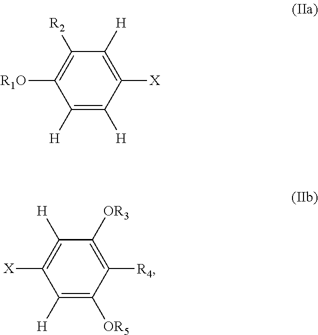
in which
the substituents R₁, R₂, R₃, R₄ and R₅ have the same meanings as defined for formula (I)
X is —I; —Br; —Cl; or —N₂,
is reacted with a compound of formula (IIIa) or (IIIb) 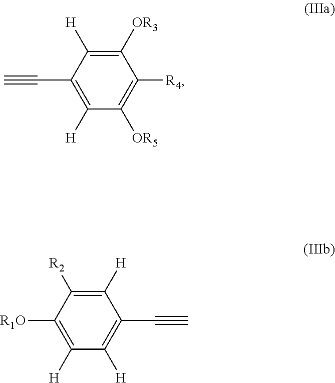
in which
the substituents R₁, R₂, R₃, R₄ and R₅ have the same meanings as defined for formula (I), and
characterized in that a heterogeneous catalytic system is used.

2. A process according to claim 1, wherein a compound of formula (IIa) is reacted with a compound of formula (IIIa).

3. A process according to claim 1, wherein a compound of formula (IIb) is reacted with a compound of formula (IIIb).

4. A process according to claim 1, wherein
R₁ is H; —CH₃; —CH₂CH₃, or —CH₂-phenyl;
R₂ is H or OR′₂, wherein R′₂ is —CH₃ or —CH₂CH₃;
R₃ is H; —CH₃; —CH₂CH₃, or —CH₂-phenyl;
R₄ is H or OR′₄, wherein R′₄ is —CH₃ or —CH₂CH₃; and
R₅ is H; —CH₃; —CH₂CH₃, or —CH₂-phenyl.

5. A process according to claim 1, wherein
R₁ is H; —CH₃; or —CH₂CH₃;
R₂ is H or OR′₂, wherein R′₂ is —CH₃ or —CH₂CH₃;
R₃ is H; —CH₃; or —CH₂CH₃;
R₄ is H or OR′₄, wherein R′₄ is —CH₃ or —CH₂CH₃; and
R₅ is H; —CH₃; or —CH₂CH₃;

6. A process according to claim 1 for a compound of formula (Ia) 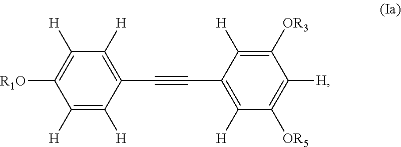
wherein
R₁, R₃ and R₅ are independently from each other H; linear, branched or cyclic C₁-C₆-alkyl; tetrahydropyryl or —CH₂-phenyl.

7. A process according to claim 1 for a compound of formula (Ib)
(Ib) 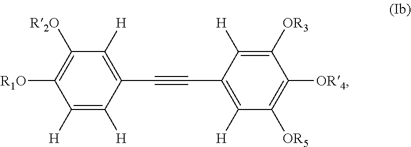
wherein
R₁, R′₂, R₃, R′₄ and R₅ are independently from each other H; linear, branched or C₁-C₆-alkyl; tetrahydropyryl or —CH₂-phenyl.

8. A process according to claim 1, wherein the catalytic system is a heterogeneous system with catalysts on a carrier.

9. A process according to claim 8, wherein the catalytic system is chosen from the group consisting of Pd/BaSO₄, Pd/CaCO₃, Pd/Al₂O₃, Pd/TiO₂, Pd/SiO₂, Pd/ZnO and Pd/C.

10. A process for the production of stilbenes of formula (IV) 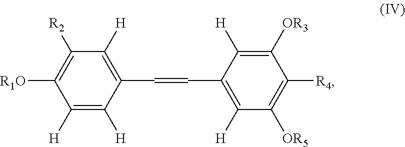
wherein
the substituents R₁, R₂, R₃, R₄ and R₅ have the meanings as defined in claim 1 by hydrogenation of compounds of formula (I) as defined in claim 1,
characterized in that a heterogeneous catalytic system comprising palladium and lead (Pb) on calcium carbonate is used.

11. A process according to claim 10, which is carried out solvent free.
